(19) United States
(12) Patent Application Publication
(10) Pub. No.: US 2012/0264929 A1
(43) Pub. Date: Oct. 18, 2012

(54) METHOD FOR PRODUCING N-ACETYL-D-GLUCOSAMINE

(76) Inventors: Mahesh Devdhar, Navi Mumbai (IN); Trupti Darge, Navi Mumbai (IN)

(21) Appl. No.: 12/665,837

(22) PCT Filed: Nov. 23, 2009
(86) PCT No.: PCT/EP2009/008320

§ 371 Date: Feb. 16, 2010

(51) Int. Cl. C07H 1/00; C07H 1/06; C07H 5/06

(52) U.S. Cl. 536/55.2

(57) ABSTRACT

The present invention relates to a novel process for producing N-acetyl-D-Glucosamine from Glucosamine hydrochloride. More particularly this invention pertains to a novel and simple environmentally acceptable process for producing glucosamine base solution in water, with very low halide content. Glucosamine base in solution with >95% chloride ion removal is further reacted with acylating agent to produce N-acetyl-D-Glucosamine with >99% purity and chloride content of <0.1%.

### CROSS-REFERENCE TO RELATED APPLICATIONS

This application is a 35 U.S.C. §371 national phase entry of PCT/EP2009/008320, filed Nov. 23, 2009, which is incorporated herein by reference.

### FIELD OF THE INVENTION

The present invention relates to a novel method for producing N-acetyl-D-Glucosamine from Glucosamine hydrochloride. More particularly this invention pertains to a novel, simple process for producing glucosamine base solution, with very low halide content. Glucosamine base in solution with >95% chloride ion removal is further reacted with acylating agent to produce N-acetyl-D-Glucosamine with >99% purity and chloride content of <0.1%.

### BACKGROUND OF THE INVENTION

N-acetyl-D-Glucosamine (NAG) is an amino-sugar, which is an important precursor for building blocks of glycoproteins, glycoasaminoglycans and glycolipids found in structural material of the cell. NAG has a therapeutic potential for treatment of various ailments such gastritis, inflammatory bowel disease, diverticulitis, in regeneration of cartilage and to stop progress of osteoarthritis.

Currently N-acetyl-D-Glucosamine is commercially made by any one of the following three processes, broadly classified such as chemical processes, or enzymatic processes or fermentation processes.

In U.S. Pat. No. 2,792,388, discloses process where in Glucosamine hydrochloride suspension in alcohol (such as methanol or ethanol) is treated with either triethyl amine or diethyl amine and acetic anhydride to obtain NAG.

U.S. Pat. No. 7,511,134, discloses a process wherein N-acetyl-D-Glucosamine is prepared by reaction of acetic anhydride with Glucosamine base (purity of >99% and halide content <0.01%) suspension in diluents comprising of C₁-C₄ alcohol. The halide free Glucosamine base is prepared by reaction of Glucosamine hydrochloride with Lithium base in diluents comprising of C₁-C₄ alcohol.

N-acetyl-D-Glucosamine may also be prepared by enzymatic processes and Microbial fermentation processes. For example, U.S. Pat. No. 5,262,310 discloses and claims a process where in chitin-containing material is heat-treated in organic solvent, or in a solvent with water, and then a beta-1,4 glycoside decomposing enzyme is added for decomposing the chitin-containing material by enzymatic reaction.

Japanese Patent No. 5,084,087 discloses the release of N-acetyl-D-glucosamine by treating a polysaccharide with acid chitinase induced with ethylene in azuki bean plant.

U.S. Pat. No. 5,998,173 describes the release of N-acetyl-D-Glucosamine by enzymatic hydrolysis of chitin of crustacean shells.

U.S. Pat. No. 6,693,188 discloses a method of preparing N-acetyl-D-Glucosamine from fungal biomass utilizing enzymes.

U.S. Pat. No. 7,33,2304 discloses a biosynthetic method that utilizes fermentation process with genetically modified microorganisms to produce D-Glucosamine and N-acetyl-D-Glucosamine.

The chemical methods for the production of N-acetyl-D-Glucosamine as described in U.S. Pat. No. 2,792,388 and U.S. Pat. No. 751,134, utilizes D-Glucosamine hydrochloride as a starting material that is converted to Glucosamine base by reacting with either diethylamine/triethylamine or Lithium hydroxide in organic solvents.

The chemical methods of the state of the art have the disadvantage that they use expensive and/or toxic reagents. Although the use of expensive and/or toxic reagents and their subsequent disposal may not cause problems on laboratory scale method of production, such methods can hardly be up scaled to large scale industrial production. Since in recent years regulations in environmental law have become more and more restrictive the need for simple environmental friendly production methods has very much increased.

### SUMMARY OF THE INVENTION

The problem of the present invention is to provide an economical, environmental friendly method for the production of pure N-acetyl-D-glucosamine usable in middle or large scale industrial production.

The problem is solved by a method for the production of N-acetyl-D-glucosamine according to the invention. Preferred embodiments are defined herein.

### DETAILED DESCRIPTION OF THE PREFERRED EMBODIMENTS

The method for the production of N-acetyl-D-glucosamine according to the present invention comprises the outlined below.

In a step a) glucosamine base solution is prepared from Glucosamine hydrochloride solution in water. Chloride ions are removed by weakly basic anion exchanger resin in —OH⁻ form.

In a step b) an acylating agent is added in stoichiometric quantity to the glucosamine base solution obtained in step a) while maintaining pH at 4-6 with sodium carbonate solution. The reaction temperature is maintained between 20-30° C.

In a step c) cations are removed from the solution obtained in step b) with weakly acidic cation exchanger resin in —H⁺ form.

In a step d) Recovery of N-acetyl-D-Glucosamine solution is recovered from the solution obtained at step c) by evaporation of water under vacuum at temperature below 35° C.

Glucosamine base solution in water is prepared without using any solvents or toxic material such as diethylamine or triethylamine or lithium hydroxide. The glucosamine base solution has low halide content since more than 95% of the halide is removed. Also the further steps of the method for the production of N-acetyl-D-glucosamine avoid toxic reagents. The weakly basic anion exchanger and the weakly acidic cation exchanger can both be regenerated and reused for many production cycles.

The method for the production of N-acetyl-D-glucosamine according to the present invention is simple and very environmentally friendly. N-acetyl-D-glucosamine is obtained in good yield, an excellent purity of more than 99%. It has a very low halide (such as chloride) content and is almost free of metal ions (such as sodium).

A method is described for the economical productions of halide free N-acetyl-D-glucosamine (NAG) that utilizes weakly basic anion exchanger resin for removal halide ions. Relatively halide free glucosamine base solution in water is immediately reacted with acetic anhydride to produce N-acetyl-D-glucosamine. Before recovering NAG, reacted solution is passed through weakly acidic cation exchanger to remove most of metal cations from it. Crude NAG solution is then concentrated by distillation under vacuum to remove water and residual acetic acid. If desired crude NAG solution may be decolorized by stirring with activated charcoal. Decolorized and filtered NAG solution, is then concentrated by distillation under vacuum to <35% moisture content. Crude NAG is suspended and stirred in methanol at less than 10° C. for 30 minutes. The crystalline NAG is filtered and further washed with several portions of chilled methanol, and then dried by conventional methods. The N-acetyl-D-Glucosamine prepared by method of invention will have purity >99%, melting point in the range of 200-210° C. and chloride content of <0.1%. This material showed no darkening on incubation at 105° C. for 2 hours.

Glucosamine base content in solution was estimated by colorimetric method as described by Cessi (Cessi C. et al. Biochem. J. (1960) 77, 508-510) and originally reported by Elson and Morgan (Elson, L. A. & Morgan, W. T. J. Biochem. J.(1933), 27,1824).

In a preferred embodiment of the method for the production of N-acetyl-D-glucosamine, water used is in an amount of 3 to 5 parts by weight per weight of glucosamine hydrochloride.

In a further preferred embodiment a temperature of the glucosamine hydrochloride solution is maintained at 30-35° C.

In another preferred embodiment the anion exchanger resin used is a weakly basic ion exchange resin that is packed in a suitable column. The weakly basic anion exchanger resin is taken in such an amount that its total ion exchange capacity in milli-equivalents (mEq) is 2 to 4 times the number of mmol of the glucosamine hydrochloride to be passed through it.

In a further preferred embodiment the glucosamine hydrochloride solution is passed through the anion exchanger resin column at a rate of 1-2 bed volume per hour. The anion exchanger resin column is given 0.5 bed volume of water washing after passing the glucosamine hydrochloride solution through it.

In a preferred embodiment the acylating agent used is acetic anhydride in ratio of 1.1 moles to 1.2 moles per mole of glucosamine base in solution. The addition of the total quantity of the acylating agent is done over a period of 40-60 minutes, whereby the temperature of an acylation reaction is maintained at 20-30° C. and the pH of the acylation reaction is maintained at 4 to 6 by addition of 250 w/v sodium carbonate solution in water.

In another preferred embodiment the cation exchanger resin used is a weakly acidic ion exchanger resin packed in a suitable column and the weakly acidic cation exchanger resin is taken in such an amount that its total ion exchange capacity in mEq is 4 to 6 times the number of mmol of the glucosamine hydrochloride passed through it. That is, the amount of cation exchanger resin is based on the number of mmol of glucosamine hydrochloride initially taken for the method.

In a preferred embodiment a solution of N-acetyl-D-glucosamine is passed through cation exchange resin column at a rate of 1-2 bed volume per hour. The cation exchanger resin column is given 0.75 bed volume of water washing after passing the reaction solution through it.

In a further preferred embodiment the solution of N-acetyl-D-glucosamine is concentrated by evaporation of water under vacuum at a temperature below 35° C. The concentrating is continued until a crude crystallized NAG has moisture content of less than 35% by weight.

In another preferred embodiment the crude crystallized NAG is re-dissolved in two parts of water, is again re-concentrated to remove residual acetic acid from the crude crystallized NAG. The crude crystallized NAG is then suspended in methanol and filtered off.

The following examples serve to more fully describe the manner of making and using the above-described inventions, as well as to set forth the best modes contemplated for carrying out various aspects of the invention. It is to be understood that these examples in no way serve to limit the true scope of this invention, but rather, are presented for illustrative purposes only. It will be understood that all proportions are in parts by weight, unless otherwise indicated.

### Example 1

In two separate glass columns, 480 ml weakly basic anion exchanger Amberlite IRA-67 resin (product of Rohm and Hass) capacity 1.6 mEq/ml and 300 ml of weakly acidic cation exchanger Amberlite IRC 76 resin (product of Rohm and Hass) capacity 4.2 mEq/ml, were packed and regenerated as per known process of prior art.

Glucosamine hydrochloride (GLH) 60.0 g was dissolved in 240 ml (4 parts) of purified water at 30-35° C. Resultant clear solution had initial pH of 2.5 and was charged to anion exchanger column at a rate of 480 ml (one bed volume) per hour. Initial void volume (200 ml) comprising of water was discarded. The product stream with glucosamine base in solution was detected by change in refractive index and was collected in reactor, along with 240 ml (0.5 bed volume) of purified water washing passed though the column. The total volume including washing was 480 ml. Glucosamine base content in solution was estimated by colorimetric method described above. The yield of Glucosamine base was 42.4 g (85.1%). The glucosamine base solution was cooled to 20-25° C. Acetic anhydride (31.2 g) was added drop wise over period of 45 minutes and temperature of exothermic reaction was maintained at 20-25° C. The reaction pH was continuously controlled in the range of 4 to 6 by addition of 25% Sodium carbonate solution. Acetic anhydride addition was completed in 45 minutes and stirring was continued for further 15 minutes.

The reaction solution was then passed through weakly acidic cation exchanger column at feed rate of 480 ml (1.6 bed volume) per hour. Initial void volume (150 ml) comprising of water was discarded. The product stream with N-acetyl-D-glucosamine in solution was detected by change in refractive index and was collected along with 150 ml of purified water washing passed through column. NAG solution was then concentrated by distillation under vacuum (0.1 mm Hg) at temperature below 35° C. The crude NAG (75 g) with 30% moisture content was dissolved in 150 ml of purified water and solution was decolorized by activated charcoal treatment. Decolorized and filtered NAG solution was concentrated again to less than 30% moisture content. The NAG crystals were then suspended in two parts by weight of methanol and stirred for 30 minutes at temperature below 10° C. and then kept at same temperature for 6 hours. Filtered NAG crystals were further washed with chilled methanol and then dried under vacuum at 50° C. The yield of N-acetyl-D-Glucosamine was 42.6 g (69.2%), with melting point of 204-205° C. The material showed no darkening on incubation at 105° C. for 2 hours.

### Example 2

In two separate columns, 40 L weakly basic anion exchanger Indion-860 resin (product of Ion Exchange India Ltd, India) capacity 1.7 mEq/ml and 25 L of weakly acidic cation exchanger Indion-236 resin (product of Ion Exchange India Ltd, India) capacity 4.1 mEq/ml, were packed and regenerated as per known process of prior art.

Glucosamine hydrochloride (GLH) 5.0 kg was dissolved in 20.0L (4 parts) of purified water at 30-35° C. Resultant clear solution was passed through anion exchanger column at a rate of 40 L (one bed volume) per hour. Initial void volume (15 L) comprising of water was discarded. The product stream with glucosamine base in solution was detected by change in refractive index and was collected in reactor, along with 20 L (0.5 bed volume) of purified water washing passed though the column. The total volume including washing was 43 L.

Glucosamine base content in solution was estimated by colorimetric method described above. The yield of Glucosamine base was 3.3 kg (80%). The glucosamine base solution was cooled to 20-25° C. Acetic anhydride (2.7 kg) was added drop wise over period of 60 minutes and temperature of exothermic reaction was maintained at 20-25° C. The reaction pH was continuously controlled in the range of 4 to 6 by addition of 25% Sodium carbonate solution. Acetic anhydride addition was completed in 60 minutes and stirring was continued for further 15 minutes.

The reaction solution was then passed through weakly acidic cation exchanger column at feed rate of 36 L (1.4 bed volume) per hour. Initial void volume (9.0 L) comprising of water was discarded. The product stream with N-acetyl-D-glucosamine in solution was detected by change in refractive index and was collected along with 12 L (0.5 bed volume) of purified water washing passed through column. NAG solution was then concentrated by distillation under vacuum (0.1 mm Hg) at temperature below 35° C. The crude NAG (5.8 Kg) with 30% moisture content was dissolved in 12.0 L of purified water and solution was decolorized by activated charcoal treatment. Decolorized and filtered NAG solution was concentrated again to less than 30% moisture content. The NAG crystals were then suspended in two parts by weight of methanol and stirred for 30 minutes at temperature below 10° C. and then kept at same temperature for 6 hours. Filtered NAG crystals were further washed with chilled methanol and then dried under vacuum at 50° C. The yield of N-acetyl-D-Glucosamine was 3.5 kg (68%), with melting point of 202-204° C. and material showed no darkening on incubation at 105° C. for 2 hours.

1. A method for producing N-acetyl-D-glucosamine which comprises steps of:
a) preparation of a glucosamine base solution from glucosamine hydrochloride solution in water whereby chloride ions are removed by a weakly basic anion exchanger resin in —OH⁻ form;
b) addition of an acylating agent in a stoichiometric quantity to the glucosamine base solution obtained in step a) while maintaining pH at 4-6 with sodium carbonate solution and while the reaction temperature is maintained between 20-30° C.;
c) removal of cations from a solution obtained in step b) with a weakly acidic cation exchanger resin in —H⁺ form; and
d) recovery of an N-acetyl-D-glucosamine solution obtained in step c) by evaporation of water under vacuum at temperature below 35° C.

2. The method of claim 1 wherein water used is in an amount of 3 to 5 parts by weight per weight of glucosamine hydrochloride.

3. The method of claim 1 wherein a temperature of the glucosamine hydrochloride solution is maintained at 30-35° C.

4. The method of claim 1 wherein the anion exchanger resin used is a weakly basic ion exchange resin packed in a suitable column and the weakly basic anion exchanger resin is taken in such an amount that its total ion exchange capacity in milli-equivalents (mEq) is 2 to 4 times the number of mmol of the glucosamine hydrochloride to be passed through it.

5. The method of claim 4 wherein the glucosamine hydrochloride solution is passed through the anion exchanger resin column at a rate of 1-2 bed volume per hour and the anion exchanger resin column is given 0.5 bed volume of water washing after passing the glucosamine hydrochloride solution through it.

6. The method of claim 1 wherein the acylating agent used is acetic anhydride in ratio of 1.1 moles to 1.2 moles per mole of glucosamine base in solution and the addition of the total quantity of the acylating agent is done over a period of 40-60 minutes, the temperature of an acylation reaction is maintained at 20-30° C. and the pH of the acylation reaction is maintained at 4 to 6 by addition of 25% w/v sodium carbonate solution in water.

7. The method of claim 2 wherein the cation exchanger resin used is a weakly acidic ion exchanger resin packed in a suitable column and the weakly acidic cation exchanger resin is taken in such an amount that its total ion exchange capacity in mEq is 4 to 6 times the number of mmol of the glucosamine hydrochloride passed through it.

8. The method of claim 6 wherein a solution of N-acetyl-D-glucosamine is passed through cation exchange resin column at a rate of 1-2 bed volume per hour and wherein the cation exchanger resin column is given 0.75 bed volume of water washing after passing the reaction solution through it.

9. The method of claim 8 wherein the solution of N-acetyl-D-glucosamine is concentrated by evaporation of water under vacuum at a temperature below 35° C. and concentrating is continued until a crude crystallized NAG has a moisture content of less than 35% by weight,

10. The method of claim 9 wherein the crude crystallized NAG is re-dissolved in two parts of water is again re-concentrated to remove residual acetic acid from the crude crystallized NAG and the crude crystallized NAG is then suspended in methanol and filtered off.
